(19) United States
(12) Patent Application Publication
(10) Pub. No.: US 2022/0107966 A1
(43) Pub. Date: Apr. 7, 2022

(54) SYSTEM AND METHOD FOR CREATING SHARED EXPERIENCES NETWORKS

(71) Applicant: Tomas Santos-Alejandro, Panama City, FL (US)

(72) Inventor: Tomas Santos-Alejandro, Panama City, FL (US)

(21) Appl. No.: 17/491,160

(22) Filed: Sep. 30, 2021

Related U.S. Application Data
(60) Provisional application No. 63/086,351, filed on Oct. 1, 2020.

(51) Int. Cl. G06F 16/28; G06F 16/2457; G16H 50/20; G06K 9/72; G06Q 50/00

(52) U.S. Cl. CPC G06F 16/288; G06F 16/2457; G06Q 50/01; G06K 9/723; G16H 50/20

(57) ABSTRACT

A system and methods for creating correlation nodes is provided. The system generally comprises a computing device having a user interface, processor operably connected to the computing device, power supply, and non-transitory computer-readable medium coupled to the processor and having instructions stored thereon. The system and method are designed to take correlation data of a PoV profile and create correlation nodes. The system may then weight the correlation nodes of the PoV profile and connect the PoV profile with other PoV profiles based on said correlation nodes. Among other things, this technique may be used to create shared experience networks using individual experiences and historical narratives, allowing the system to connect users without the need of a common user connection.

### CROSS REFERENCES TO RELATED APPLICATIONS

This application claims priority to U.S. Provisional Patent Application Ser. No. 63/086,351 filed on Oct. 1, 2020, the entirety of which is incorporated herein by reference.

### FIELD OF THE DISCLOSURE

The subject matter of the present disclosure refers generally to a system and method for creating shared experiences networks using individual experiences and historical narratives.

### BACKGROUND

Social media is an almost effortless way to connect with like-minded individuals. As such, social media has become one of the primary ways in which many people find new friends. Social media is also a great way to build communities, such as fanbases of sports teams, and to promote noble causes, such as social welfare. The organization of social groups is particularly beneficial for the mental health of individuals by reducing social isolation many people experience in our modern society. Further, social media is also now one of the main ways in which businesses advertise their business since it reaches multiple demographics and is cheaper than conventional advertising methods, such as television commercials. It also helps businesses grow in ways that allow it to better understand its employees and customer base, allowing them to be more efficient both internally and externally.

Traditionally, social media platforms have linked users based on contacts within their social network. For instance, social media platforms may recommend other users to a first user based on a related contact within the other users' and the first user's contact list. Social media platforms also may attempt to connect users based on groups they may be a part of. For instance, users who are in the same mountain climbing group but not connected to each other's social network in any other meaningful way may be recommended to one another via the social media platform as a potential social contact. Though socially connecting users using this method can work, it only works if there is an existing connection that directly links one user to another. Users who may share similar interests but have no connections in the social media platform will have a difficult time connecting unless some other means of social contact generation is used.

Therefore, there is a need in the art for a system and method that recognizes potential social contacts based on users' documented experiences.

### SUMMARY

A system and methods for creating correlation nodes is provided. In one aspect, the system creates correlation nodes based on correlation data and then connects those correlation nodes 435 to other correlation nodes based on different correlation data. In another aspect, the system creates correlation nodes from correlation data of users and links said users based on commonality points between those correlation nodes. Generally, the system uses context and time frames to make connections in data that may be difficult to perceive using more conventional methods. In a preferred embodiment, the system allows users to connect to one another without the need for the users to have pre-existing social contacts within their social network. The system generally comprises a computing device, processor operably connected to the computing device, power supply, and non-transitory computer-readable medium coupled to the processor and having instructions stored thereon. Correlation nodes may be created by the system using correlation data within a point of view (PoV) profile. In some preferred embodiments, the system may further comprise groups. For instance, a user may add themselves to a group via a user interface, which may allow a user to limit correlation data from which correlation nodes are derived.

The foregoing summary has outlined some features of the system and method of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the system and method disclosed herein. Those skilled in the pertinent art should also realize that such equivalent designs or modifications do not depart from the scope of the system and method of the present disclosure.

### DESCRIPTION OF THE DRAWINGS

These and other features, aspects, and advantages of the present disclosure will become better understood with regard to the following description, appended claims, and accompanying drawings where:

FIG. 1 is a diagram of an example environment in which techniques described herein may be implemented.

FIG. 2 is a diagram of an example environment in which techniques described herein may be implemented.

FIG. 3 is a diagram of an example environment in which techniques described herein may be implemented.

FIG. 4 is a diagram illustrating a system embodying features consistent with the principles of the present disclosure.

FIG. 5 is a diagram illustrating a user interface embodying features consistent with the principles of the present disclosure.

FIG. 6 is a diagram illustrating a user interface embodying features consistent with the principles of the present disclosure.

FIG. 7 is a diagram illustrating a user interface embodying features consistent with the principles of the present disclosure.

FIG. 8 is a diagram illustrating a system embodying features consistent with the principles of the present disclosure.

FIG. 9 a diagram illustrating a flow chart illustrating certain method steps of a method embodying features consistent with the principles of the present disclosure.

FIG. 10 a diagram illustrating a flow chart illustrating certain method steps of a method embodying features consistent with the principles of the present disclosure.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises” and grammatical equivalents thereof are used herein to mean that other components, steps, etc. are optionally present. For example, a system “comprising” components A, B, and C can contain only components A, B, and C, or can contain not only components A, B, and C, but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

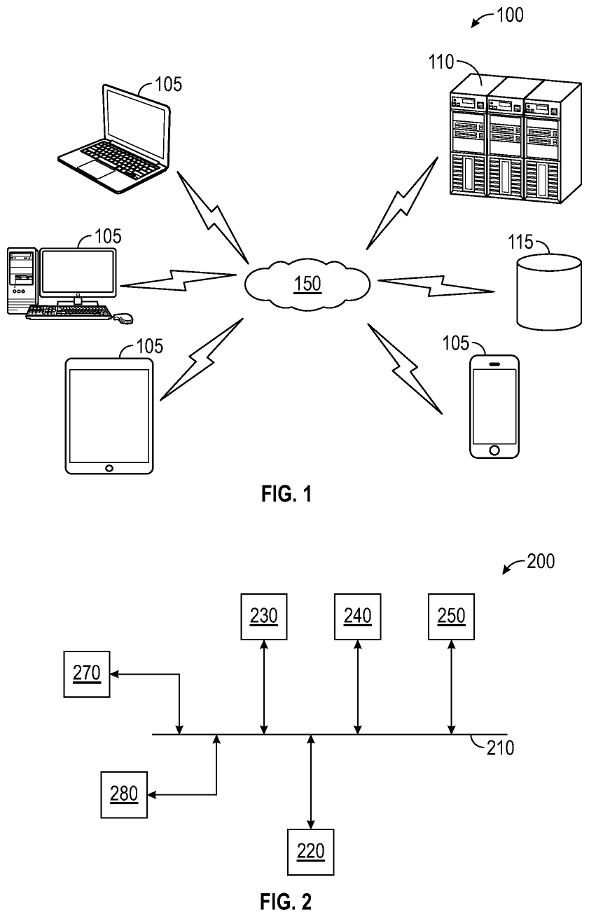

FIG. 1 depicts an exemplary environment 100 of the system 400 consisting of clients 105 connected to a server 110 and/or database 115 via a network 150. Clients 105 are devices of users 405 that may be used to access servers 110 and/or databases 115 through a network 150. A network 150 may comprise of one or more networks of any kind, including, but not limited to, a local area network (LAN), a wide area network (WAN), metropolitan area networks (MAN), a telephone network, such as the Public Switched Telephone Network (PSTN), an intranet, the Internet, a memory device, another type of network, or a combination of networks. In a preferred embodiment, computing entities 200 may act as clients 105 for a user 405. For instance, a client 105 may include a personal computer, a wireless telephone, a streaming device, a “smart” television, a personal digital assistant (PDA), a laptop, a smart phone, a tablet computer, or another type of computation or communication interface 280. Servers 110 may include devices that access, fetch, aggregate, process, search, provide, and/or maintain documents. Although FIG. 1 depicts a preferred embodiment of an exemplary environment 100 for the system 400, in other implementations, the exemplary environment 100 may contain fewer components, different components, differently arranged components, and/or additional components than those depicted in FIG. 1. Alternatively, or additionally, one or more components of the exemplary environment 100 may perform one or more other tasks described as being performed by one or more other components of the exemplary environment 100.

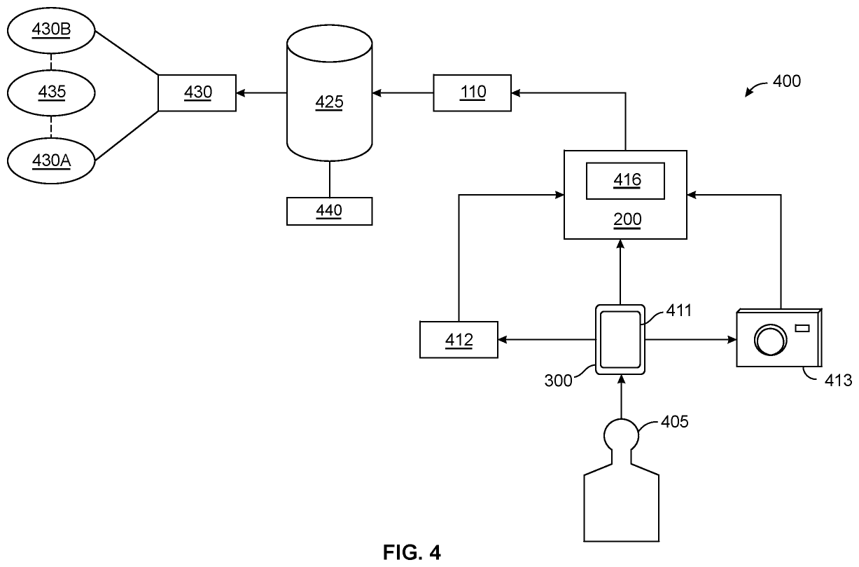

Some embodiments of the system 400 may comprise a server 110. A server 110 may be a search server, a document indexing server, and general web server. Servers 110 may be separate entities performing different functions or similar functions. For instance, two or more servers 110 may be implemented to work as a single server 110 performing the same tasks. Alternatively, one server 110 may perform the functions of multiple servers 110. For instance, a single server 110 may perform the tasks of a web server and an indexing server. Although represented as a single server 110 in FIG. 4, it is understood that multiple servers 110 may be used to operably connect the processor 220 to the database 115. The processor 220 may be operably connected to the server 110 via wired or wireless connection. Search servers may include one or more computing devices 300 designed to implement a search engine, such as a documents/records search engine, general webpage search engine, etc. Search servers, for example, may include one or more web servers to receive search queries and/or inputs from users 405, search one or more databases 115 in response to the search queries and/or inputs, and provide documents or information, relevant to the search queries and/or inputs, to users 405. In some implementations, search servers may include a web search server that may provide webpages to users 405, where a provided webpage may include a reference to a web server at which the desired information and/or links is located. The references, to the web server at which the desired information is located, may be included in a frame and/or text box, or as a link to the desired information/document.

Document indexing servers may include one or more computing devices 300 designed to index documents available through networks. Document indexing servers may access other servers 110, such as web servers that host the system 400, to index the display data. In some implementations, document indexing servers may index documents/records stored by other servers 110 connected to the network. Document indexing servers may, for example, store and index correlation data 430A, correlation nodes 435, and other information relating to social contacts. Web servers may include servers 110 that provide webpages to clients. For instance, the webpages may be HTML-based webpages. A web server may host one or more websites. A website, as the term is used herein, may refer to a collection of related webpages. Frequently, a website may be associated with a single domain name, although some websites may potentially encompass more than one domain name. The concepts described herein may be applied on a per-website basis. Alternatively, in some implementations, the concepts described herein may be applied on a per-webpage basis.

As used herein, a database 115 refers to a set of related data and the way it is organized. Access to this data is usually provided by a database management system (DBMS) consisting of an integrated set of computer software that allows users 405 to interact with one or more databases 115 and provides access to all of the data contained in the database 115. The DBMS provides various functions that allow entry, storage and retrieval of large quantities of information and provides ways to manage how that information is organized. Because of the close relationship between the database 115 and the DBMS, as used herein, the term database 115 refers to both a database 115 and DBMS.

FIG. 2 is an exemplary diagram of a client 105, server 110, and/or or database 115 (hereinafter collectively referred to as “computing entity 200”), which may correspond to one or more of the clients 105, servers 110, and databases 115 according to an implementation consistent with the principles of the invention as described herein. The computing entity 200 may comprise a bus 210, a processor 220, memory 304, a storage device 250, a peripheral device 270, and a communication interface 280 (such as wired or wireless communication device). The bus 210 may be defined as one or more conductors that permit communication among the components of the computing entity 200. The processor 220 may be defined as logic circuitry that responds to and processes the basic instructions that drive the computing entity 200. Memory 304 may be defined as the integrated circuitry that stores information for immediate use in a computing entity 200. A peripheral device 270 may be defined as any hardware used by a user 405 and/or the computing entity 200 to facilitate communicate between the two. A storage device 250 may be defined as a device used to provide mass storage to a computing entity 200. A communication interface 280 may be defined as any transceiver-like device that enables the computing entity 200 to communicate with other devices and/or computing entities 200.

The bus 210 may comprise a high-speed interface 308 and/or a low-speed interface 312 that connects the various components together in a way such they may communicate with one another. A high-speed interface 308 manages bandwidth-intensive operations for computing device 300, while a low-speed interface 312 manages lower bandwidth-intensive operations. In some preferred embodiments, the high-speed interface 308 of a bus 210 may be coupled to the memory 304, display 316, and to high-speed expansion ports 310, which may accept various expansion cards such as a graphics processing unit (GPU). In other preferred embodiments, the low-speed interface 312 of a bus 210 may be coupled to a storage device 250 and low-speed expansion ports 314. The low-speed expansion ports 314 may include various communication ports, such as USB, Bluetooth, Ethernet, wireless Ethernet, etc. Additionally, the low-speed expansion ports 314 may be coupled to one or more peripheral devices 270, such as a keyboard, pointing device, scanner, and/or a networking device, wherein the low-speed expansion ports 314 facilitate the transfer of input data from the peripheral devices 270 to the processor 220 via the low-speed interface 312.

The processor 220 may comprise any type of conventional processor or microprocessor that interprets and executes computer readable instructions. The processor 220 is configured to perform the operations disclosed herein based on instructions stored within the system 400. The processor 220 may process instructions for execution within the computing entity 200, including instructions stored in memory 304 or on a storage device 250, to display graphical information for a graphical user interface (GUI) on an external peripheral device 270, such as a display 316. The processor 220 may provide for coordination of the other components of a computing entity 200, such as control of user interfaces 411, applications run by a computing entity 200, and wireless communication by a communication interface 280 of the computing entity 200. The processor 220 may be any processor or microprocessor suitable for executing instructions. In some embodiments, the processor 220 may have a memory device therein or coupled thereto suitable for storing the data, content, or other information or material disclosed herein. In some instances, the processor 220 may be a component of a larger computing entity 200. A computing entity 200 that may house the processor 220 therein may include, but are not limited to, laptops, desktops, workstations, personal digital assistants, servers 110, mainframes, cellular telephones, tablet computers, smart televisions, streaming devices, or any other similar device. Accordingly, the inventive subject matter disclosed herein, in full or in part, may be implemented or utilized in devices including, but are not limited to, laptops, desktops, workstations, personal digital assistants, servers 110, mainframes, cellular telephones, tablet computers, smart televisions, streaming devices, or any other similar device.

Memory 304 stores information within the computing device 300. In some preferred embodiments, memory 304 may include one or more volatile memory units. In another preferred embodiment, memory 304 may include one or more non-volatile memory units. Memory 304 may also include another form of computer-readable medium, such as a magnetic, solid state, or optical disk. For instance, a portion of a magnetic hard drive may be partitioned as a dynamic scratch space to allow for temporary storage of information that may be used by the processor 220 when faster types of memory, such as random-access memory (RAM), are in high demand. A computer-readable medium may refer to a non-transitory computer-readable memory device. A memory device may refer to storage space within a single storage device 250 or spread across multiple storage devices 250. The memory 304 may comprise main memory 230 and/or read only memory (ROM) 240. In a preferred embodiment, the main memory 230 may comprise RAM or another type of dynamic storage device 250 that stores information and instructions for execution by the processor 220. ROM 240 may comprise a conventional ROM device or another type of static storage device 250 that stores static information and instructions for use by processor 220. The storage device 250 may comprise a magnetic and/or optical recording medium and its corresponding drive.

As mentioned earlier, a peripheral device 270 is a device that facilitates communication between a user 405 and the processor 220. The peripheral device 270 may include, but is not limited to, an input device and/or an output device. As used herein, an input device may be defined as a device that allows a user 405 to input data and instructions that is then converted into a pattern of electrical signals in binary code that are comprehensible to a computing entity 200. An input device of the peripheral device 270 may include one or more conventional devices that permit a user 405 to input information into the computing entity 200, such as a controller, scanner, phone, camera, scanning device, keyboard, a mouse, a pen, voice recognition and/or biometric mechanisms, etc. As used herein, an output device may be defined as a device that translates the electronic signals received from a computing entity 200 into a form intelligible to the user 405. An output device of the peripheral device 270 may include one or more conventional devices that output information to a user 405, including a display 316, a printer, a speaker, an alarm, a projector, etc. Additionally, storage devices 250, such as CD-ROM drives, and other computing entities 200 may act as a peripheral device 270 that may act independently from the operably connected computing entity 200. For instance, a streaming device may transfer data to a smartphone, wherein the smartphone may use that data in a manner separate from the streaming device.

The storage device 250 is capable of providing the computing entity 200 mass storage. In some embodiments, the storage device 250 may comprise a computer-readable medium such as the memory 304, storage device 250, or memory 304 on the processor 220. A computer-readable medium may be defined as one or more physical or logical memory devices and/or carrier waves. Devices that may act as a computer readable medium include, but are not limited to, a hard disk device, optical disk device, tape device, flash memory or other similar solid-state memory device, or an array of devices, including devices in a storage area network or other configurations. Examples of computer-readable mediums include, but are not limited to, magnetic media such as hard disks, floppy disks, and magnetic tape; optical media such as CD ROM discs and DVDs; magneto-optical media such as optical discs; and hardware devices that are specially configured to store and perform programming instructions, such as ROM 240, RAM, flash memory, and the like.

In an embodiment, a computer program may be tangibly embodied in the storage device 250. The computer program may contain instructions that, when executed by the processor 220, performs one or more steps that comprise a method, such as those methods described herein. The instructions within a computer program may be carried to the processor 220 via the bus 210. Alternatively, the computer program may be carried to a computer-readable medium, wherein the information may then be accessed from the computer-readable medium by the processor 220 via the bus 210 as needed. In a preferred embodiment, the software instructions may be read into memory 304 from another computer-readable medium, such as data storage device 250, or from another device via the communication interface 280. Alternatively, hardwired circuitry may be used in place of or in combination with software instructions to implement processes consistent with the principles as described herein. Thus, implementations consistent with the invention as described herein are not limited to any specific combination of hardware circuitry and software.

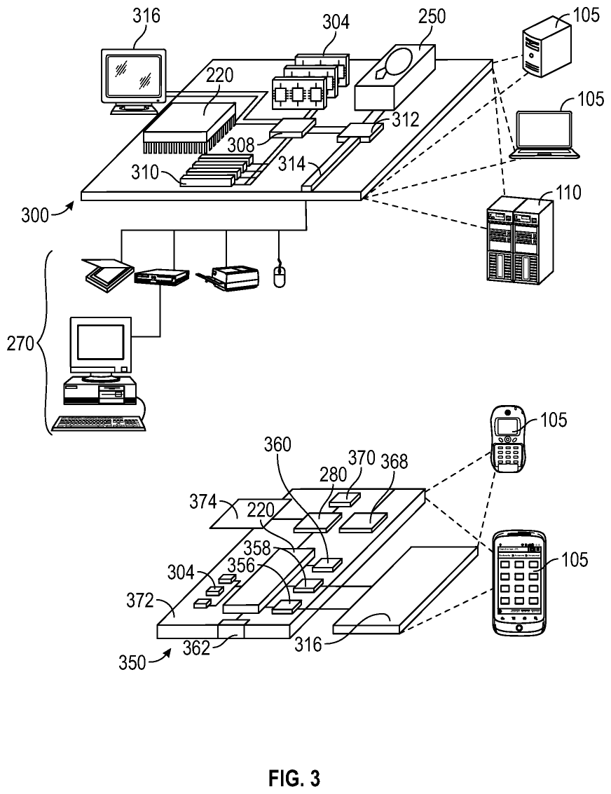

FIG. 3 depicts exemplary computing entities 200 in the form of a computing device 300 and mobile computing device 350, which may be used to carry out the various embodiments of the invention as described herein. A computing device 300 is intended to represent various forms of digital computers, such as laptops, desktops, workstations, servers 110, databases 115, mainframes, and other appropriate computers. A mobile computing device 350 is intended to represent various forms of mobile devices, such as scanners, scanning devices, personal digital assistants, cellular telephones, smart phones, tablet computers, and other similar devices. The various components depicted in FIG. 3, as well as their connections, relationships, and functions are meant to be examples only, and are not meant to limit the implementations of the invention as described herein. The computing device 300 may be implemented in a number of different forms, as shown in FIGS. 1 and 3. For instance, a computing device 300 may be implemented as a server 110 or in a group of servers 110. Computing devices 300 may also be implemented as part of a rack server system. In addition, a computing device 300 may be implemented as a personal computer, such as a desktop computer or laptop computer. Alternatively, components from a computing device 300 may be combined with other components in a mobile device, thus creating a mobile computing device 350. Each mobile computing device 350 may contain one or more computing devices 300 and mobile devices, and an entire system may be made up of multiple computing devices 300 and mobile devices communicating with each other as depicted by the mobile computing device 350 in FIG. 3. The computing entities 200 consistent with the principles of the invention as disclosed herein may perform certain receiving, communicating, generating, output providing, correlating, and storing operations as needed to perform the various methods as described in greater detail below.

In the embodiment depicted in FIG. 3, a computing device 300 may include a processor 220, memory 304 a storage device 250, high-speed expansion ports 310, low-speed expansion ports 314, and bus 210 operably connecting the processor 220, memory 304, storage device 250, high-speed expansion ports 310, and low-speed expansion ports 314. In one preferred embodiment, the bus 210 may comprise a high-speed interface 308 connecting the processor 220 to the memory 304 and high-speed expansion ports 310 as well as a low-speed interface 312 connecting to the low-speed expansion ports 314 and the storage device 250. Because each of the components are interconnected using the bus 210, they may be mounted on a common motherboard as depicted in FIG. 3 or in other manners as appropriate. The processor 220 may process instructions for execution within the computing device 300, including instructions stored in memory 304 or on the storage device 250. Processing these instructions may cause the computing device 300 to display graphical information for a GUI on an output device, such as a display 316 coupled to the high-speed interface 308. In other implementations, multiple processors and/or multiple buses may be used, as appropriate, along with multiple memory units and/or multiple types of memory. Additionally, multiple computing devices may be connected, wherein each device provides portions of the necessary operations.

A mobile computing device 350 may include a processor 220, memory 304 a peripheral device 270 (such as a display 316, a communication interface 280, and a transceiver 368, among other components). A mobile computing device 350 may also be provided with a storage device 250, such as a micro-drive or other previously mentioned storage device 250, to provide additional storage. Preferably, each of the components of the mobile computing device 350 are interconnected using a bus 210, which may allow several of the components of the mobile computing device 350 to be mounted on a common motherboard as depicted in FIG. 3 or in other manners as appropriate. In some implementations, a computer program may be tangibly embodied in an information carrier. The computer program may contain instructions that, when executed by the processor 220, perform one or more methods, such as those described herein. The information carrier is preferably a computer- readable medium, such as memory, expansion memory 374, or memory 304 on the processor 220 such as ROM 240, that may be received via the transceiver or external interface 362. The mobile computing device 350 may be implemented in a number of different forms, as shown in FIG. 3. For example, a mobile computing device 350 may be implemented as a cellular telephone, part of a smart phone, personal digital assistant, or other similar mobile device.

The processor 220 may execute instructions within the mobile computing device 350, including instructions stored in the memory 304 and/or storage device 250. The processor 220 may be implemented as a chipset of chips that may include separate and multiple analog and/or digital processors. The processor 220 may provide for coordination of the other components of the mobile computing device 350, such as control of the user interfaces 411, applications run by the mobile computing device 350, and wireless communication by the mobile computing device 350. The processor 220 of the mobile computing device 350 may communicate with a user 405 through the control interface 358 coupled to a peripheral device 270 and the display interface 356 coupled to a display 316. The display 316 of the mobile computing device 350 may include, but is not limited to, Liquid Crystal Display (LCD), Light Emitting Diode (LED) display, Organic Light Emitting Diode (OLED) display, and Plasma Display Panel (PDP), or any combination thereof. The display interface 356 may include appropriate circuitry for causing the display 316 to present graphical and other information to a user 405. The control interface 358 may receive commands from a user 405 via a peripheral device 270 and convert the commands into a computer readable signal for the processor 220. In addition, an external interface 362 may be provided in communication with processor 220, which may enable near area communication of the mobile computing device 350 with other devices. The external interface 362 may provide for wired communications in some implementations or wireless communication in other implementations. In a preferred embodiment, multiple interfaces may be used in a single mobile computing device 350 as is depicted in FIG. 3.

Memory 304 stores information within the mobile computing device 350. Devices that may act as memory 304 for the mobile computing device 350 include, but are not limited to computer-readable media, volatile memory, and non-volatile memory. Expansion memory 374 may also be provided and connected to the mobile computing device 350 through an expansion interface 372, which may include a Single In-Line Memory Module (SIM) card interface or micro secure digital (Micro-SD) card interface. Expansion memory 374 may include, but is not limited to, various types of flash memory and non-volatile random-access memory (NVRAM). Such expansion memory 374 may provide extra storage space for the mobile computing device 350. In addition, expansion memory 374 may store computer programs or other information that may be used by the mobile computing device 350. For instance, expansion memory 374 may have instructions stored thereon that, when carried out by the processor 220, cause the mobile computing device 350 perform the methods described herein. Further, expansion memory 374 may have secure information stored thereon; therefore, expansion memory 374 may be provided as a security module for a mobile computing device 350, wherein the security module may be programmed with instructions that permit secure use of a mobile computing device 350. In addition, expansion memory 374 having secure applications and secure information stored thereon may allow a user 405 to place identifying information on the expansion memory 374 via the mobile computing device 350 in a non-hackable manner.

A mobile computing device 350 may communicate wirelessly through the communication interface 280, which may include digital signal processing circuitry where necessary. The communication interface 280 may provide for communications under various modes or protocols, including, but not limited to, Global System Mobile Communication (GSM), Short Message Services (SMS), Enterprise Messaging System (EMS), Multimedia Messaging Service (MMS), Code Division Multiple Access (CDMA), Time Division Multiple Access (TDMA), Personal Digital Cellular (PDC), Wideband Code Division Multiple Access (WCDMA), IMT Multi-Carrier (CDMAX 0) , and General Packet Radio Service (GPRS), or any combination thereof. Such communication may occur, for example, through a transceiver 368. Short-range communication may occur, such as using a Bluetooth, WIFI, or other such transceiver 368. In addition, a Global Positioning System (GPS) receiver module 370 may provide additional navigation-and location-related wireless data to the mobile computing device 350, which may be used as appropriate by applications running on the mobile computing device 350. Alternatively, the mobile computing device 350 may communicate audibly using an audio codec 360, which may receive spoken information from a user 405 and covert the received spoken information into a digital form that may be processed by the processor 220. The audio codec 360 may likewise generate audible sound for a user 405, such as through a speaker, e.g., in a handset of mobile computing device 350. Such sound may include sound from voice telephone calls, recorded sound such as voice messages, music files, etc. Sound may also include sound generated by applications operating on the mobile computing device 350.

The power supply may be any source of power that provides the system 400 with power.

In one preferred embodiment, the system 400 may comprise of multiple power supplies that may provide power to the system 400 in different circumstances. For instance, the system 400 may be directly plugged into a stationary power source, such as a stationary electrical outlet, which may provide power to the system 400 so long as it remains in one place. However, the system 400 may also be connected to a mobile power supply, such as a battery, so that the system 400 may receive power even when it is not receiving power from a stationary power outlet. In this way, the system 400 may always receive power so that it may continuously create correlation nodes 435 and make connections based on said correlation nodes 435.

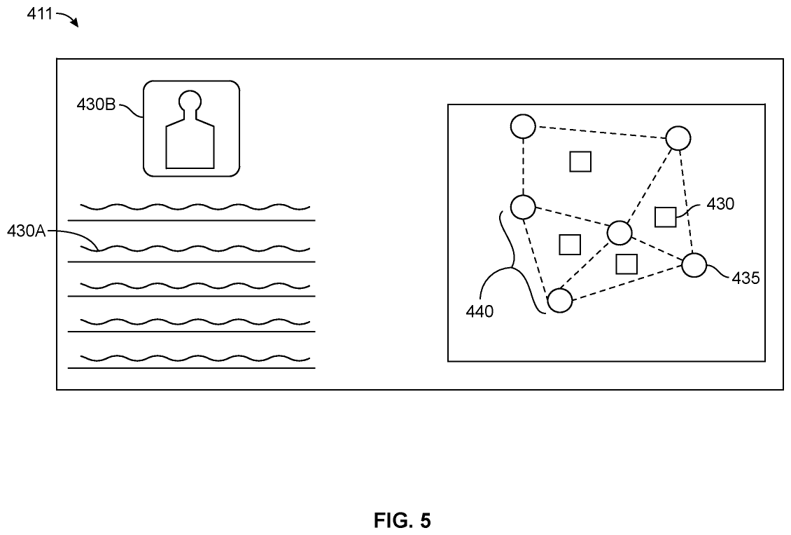

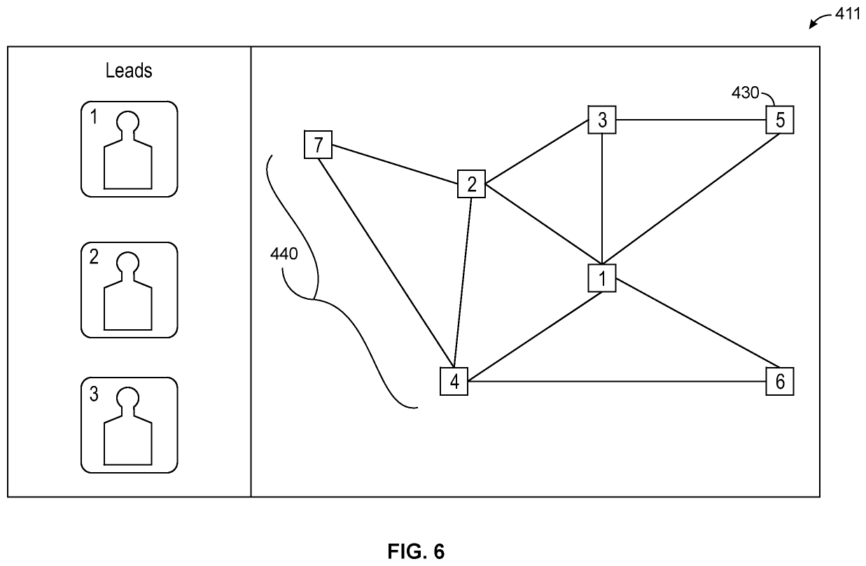

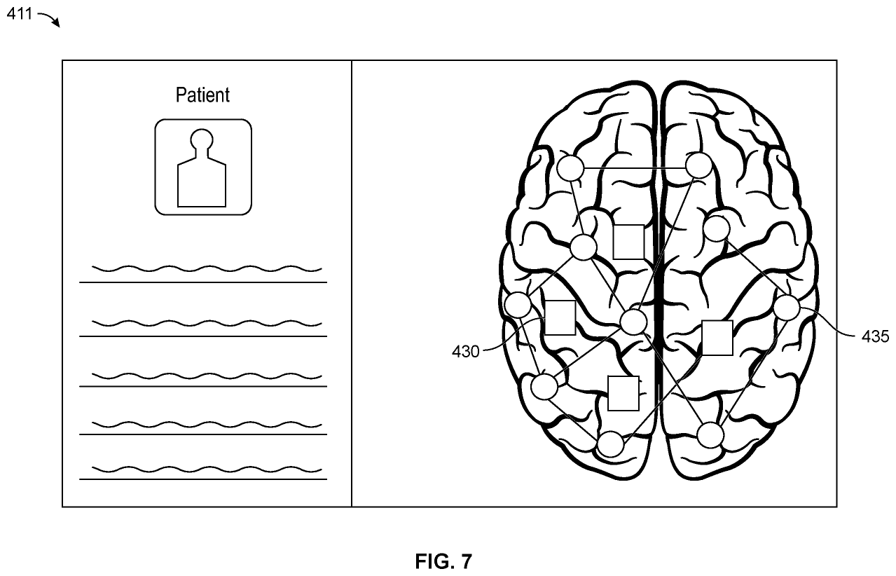

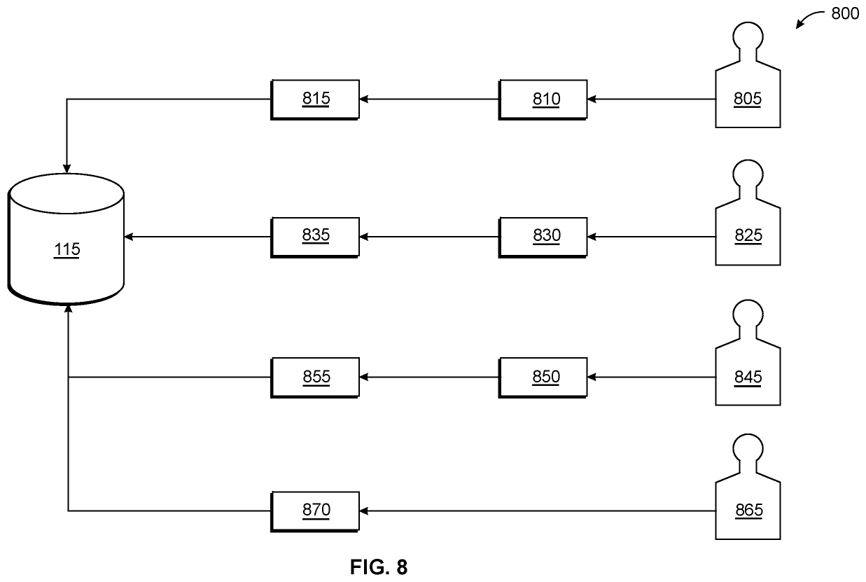

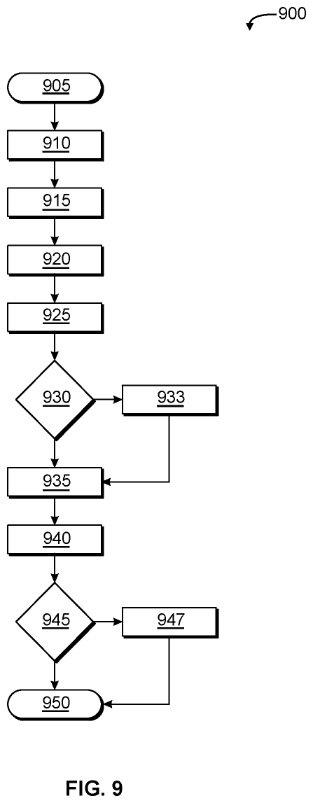

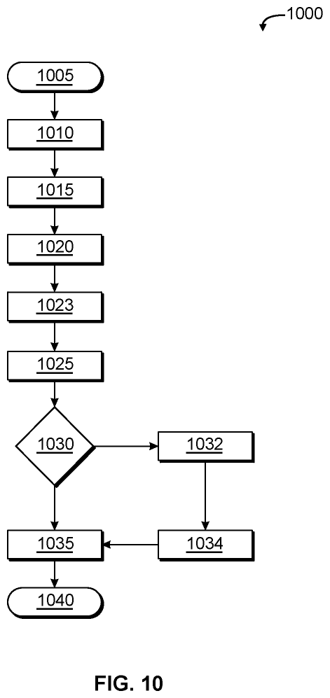

FIGS. 4-10 illustrate embodiments of a system 400 and methods that use context and time frames to make connections in data. FIG. 4 depicts a preferred embodiment of a system 400 designed to create correlation nodes 435 using correlation data. FIG. 5 illustrates a user interface 411 of an embodiment of the system 400 designed to generate social contacts using correlation nodes 435. FIG. 6 illustrates a user interface 411 of an embodiment of the system 400 that law enforcement may use to make connections in criminal case files. FIG. 7 illustrates a user interface 411 of an embodiment of the system 400 that mental health professionals may use to assist in make diagnoses and treatment protocols for patients. FIG. 8 illustrates permission levels 800 that may be utilized by the present system 400 for controlling access to content of the system 400. FIGS. 9 and 10 illustrate methods that may be carried out by the system 400. It is understood that the various method steps associated with the methods of the present disclosure may be carried out as operations by the system 400 depicted in FIG. 4.

As illustrated in FIG. 4, the system 400 generally comprises a computing device 300, processor 220 operably connected to the computing device 300, power supply, and non-transitory computer-readable medium (CRM) 416 coupled to the processor 220 and having instructions stored thereon. In one embodiment, the system 400 may comprise a display, wherein said display may present a user interface 411 of the system that may allow a user 405 to view PoV profiles 430 of social connections and/or cause the system 400 to perform an action via commands input by said user 405. In another embodiment, the system 400 may comprise a database 115 operably connected to the processor 220, which may be used to store correlation data 430A and image data 430B therein. In yet another preferred embodiment, a server 110 may be operably connected to the database 115 and processor 220, facilitating the transfer of information between the processor 220 and database 115. In yet another preferred embodiment, the system 400 may comprise a scanning device, which may allow a user 405 to input hard copies of data into the system 400 in the form of image data that may then be parsed for correlation data 430A.

The system 400 preferably transmits correlation data 430A to a display of the computing device 300 so that it may be presented to the user. In particular, the system 400 is designed to allow users 405 to control which correlation data 430A may be used to generate correlation nodes 435 as well as allow the user 405 to connect with a social contact generated by the system 400. For instance, a user 405 may select which correlation data the system 400 may use to generate social contacts via correlation nodes 435 via the user interface 411 presented in the display. For instance, as illustrated in FIG. 5, a user 405 may select which social contacts to make a connection with via the user interface 411 presented in the display.

A display may be defined as an output device that correlation data, correlation nodes 435, and social contacts may be displayed, which may include, but is not limited to, visual, auditory, cutaneous, kinesthetic, olfactory, and gustatory, or any combination thereof. Information presented via a display may be referred to as a soft copy of the information because the information exists electronically and is presented for a temporary period of time. Information stored on the CRM 416 may be referred to as the hard copy of the information. For instance, a display may present a soft copy of a visual representation of correlation nodes 435 via a liquid crystal display (LCD), wherein the hard copy of the visual representation of the correlation nodes 435 may be stored on a local hard drive. For instance, a display may present a soft copy of audio information related to why a social contact has been recommended to a user 405 via a speaker, wherein the hard copy of the audio information is stored on a flash drive. For instance, a display may present a soft copy of correlation data 430A, wherein the hard copy of the correlation data 430A is stored within a database 115. Displays may include, but are not limited to, cathode ray tube monitors, LCD monitors, light emitting diode (LED) monitors, gas plasma monitors, screen readers, speech synthesizers, haptic suits, speakers, and scent generating devices, or any combination thereof, but is not limited to these devices.

In an embodiment, the programming instructions responsible for the operations carried out by the processor 220 are stored on the CRM 416, which may be coupled to the server 110 and/or database 115, as illustrated in FIG. 4. Alternatively, the programming instructions may be stored on or included within the processor 220. Examples of non-transitory computer-readable mediums 416 include, but are not limited to, magnetic media such as hard disks, floppy disks, and magnetic tape; optical media such as CD ROM discs and DVDs; magneto-optical media such as optical discs; and hardware devices that are specifically configured to store and perform programming instructions, such as read-only memory (ROM), random access memory (RAM), flash memory, and the like. In some embodiments, the programming instructions may be stored as modules within the CRM 416.

Data within the system 400 may be stored in point of view (PoV) profiles 430. Types of profiles that may act as PoV profiles 430 include, but are not limited to, user profiles, patient profiles, case profiles, or any combination thereof. In a preferred embodiment, data is stored within the PoV profiles. Types of data that may be saved within the system 400 include, but is not limited to, correlation data and image data. Correlation data 430A may be defined as data that describes a particular event or plurality of events from a particular point of view (PoV). Image data 430B may be defined as data containing pictures encoded as binary data, which preferably contain picture elements in the form of pixels. Correlation data 430A may include, but is not limited to, name, date of birth, address, geolocation, event description, event date, or any combination thereof. Image data 430B may contain a single frame of reference or multiple frames of reference strung together to create a video. In some preferred embodiments, image data 430B may act as correlation data 430A. In other preferred embodiments, image data 430B may contain correlation data 430A that the system 400 may extract. Correlation data 430A and image data 430B may allow the system 400 to connect users 405 based on commonalities other than exiting contacts.

In one preferred embodiment, as illustrated in FIG. 5, the system 400 may comprise correlation data 430A and image data 430B belonging to particular users 405 stored in user profiles, which may allow the system 400 to create correlation nodes 435 and connect users 405 based on those correlation nodes 435. A user profile may be defined as a profile containing data about a particular user 405 including, but not limited to, identity, gender, date of birth, hobbies, profession, address, geolocation, or any combination thereof. In one preferred embodiment, user profiles may contain answers to questions posed by the system 400. These questions may be separated into categories, which the system 400 may use to help generate correlation nodes 435 for a user 405. The system 400 may separate user profiles into groups 440 and subgroups (or user roles 810, 830, 850). In a preferred embodiment, various groups 440 and subgroups of the system 400 may grant permission levels 800 that give users 405 access to data of other users 405. For instance, the user profile of a user 405 who is a paying member of a social media platform may be granted permission levels 800 that allows the paying member to access correlation data 430A pertaining to correlation nodes 435 of the other users 405. A user profile of a non-paying member who is in the paying user's 405 existing contacts (sub-group) may be granted a permission level 800 that grant the non-paying member access to the paying member's data pertaining to correlation nodes 435 but not to data pertaining to their own correlation nodes 435.

In another preferred embodiment, as illustrated in FIG. 6, the system 400 may comprise correlation data 430A and image data 430B belonging to particular cases stored in case profiles, which may allow the system 400 to create correlation nodes 435 and connect various aspects of the case. A case profile may be defined as a profile containing data about a particular case including, but not limited to, event descriptions, involved parties, backgrounds of involved parties, witness accounts, addresses, geolocations, or any combination thereof. In one preferred embodiment, case profiles may contain “thoughts,” which may be defined as notes that detectives have made about a case concerning deductions they may have made based. These “thoughts” may assist the system 400 in developing new approaches in which to view a case, which may allow the system 400 to generate new leads or make connections to past cases. In some preferred embodiments, a user 405 may limit which correlation data the system 400 may use to assist with cases. For instance, a user 405 may want to exclude the use of “thoughts” during the creation of correlation nodes 435 in order to reduce the amount of user error that may be introduced into the analysis. This may allow users 405 to approach cases from various angles, which may increase the number of cases that are successfully resolved. Therefore, in some preferred embodiments, a user interface 411 may be configured to allow a user 405 to choose which correlation data 430A is used to generate correlation nodes 435. For instance, filters may be used to filter out correlation data 430A not within a specified date range, allowing the system 400 to generate correlation nodes 435 using correlation data 430A corresponding to said specified date range.

In another preferred embodiment, the system 400 may comprise lead profiles, wherein said lead profiles contain correlation data related to a particular person of interest of a case. The system 400 create correlation nodes 435 for the case profile and the lead profile and find correlations between the two. In some preferred embodiments, the system 400 may find correlations between multiple case profiles and lead profiles associated with said case profiles. For instance, the system 400 may find correlations between murders that are years and miles apart. The system 400 may also determine that a lead profile associated with a first case profile but not a second case profile has correlations with both case profiles despite that person of interest not even being a person of interest in the second case profile. This situation can arise in situations where the evidence of the first case profile indicates that the person of interest was in the vicinity of a similar crime of the second case profile but was never even considered a person of interest due to lack of evidence. This could allow law enforcement the ability to solve many crimes where for whatever reason they were unable to acquire incriminating evidence at one time but acquired it at a later time during a seemingly unrelated incident.

In another preferred embodiment, as illustrated in FIG. 7, the system 400 may comprise correlation data 430A and image data 430B belonging to particular patients stored in patient profiles, which may allow the system 400 to assist medical professionals in making medical diagnoses and recommend treatment protocols. A patient profile may be defined as a user profile 430 containing data about a particular patient including, but not limited to, identity, gender, date of birth, journal entries, or any combination thereof. In one preferred embodiment, patient profiles may contain answers to questions posed by the system 400 and/or medical professional that may inquire into the general well-being of the patient. These answers to these questions may assist the system 400 in reaching diagnosis pertaining to the patient's mental health as well as recommend treatment protocols from which a physician may use to treat the patient.

In one preferred embodiment, the system 400 may create a condition profile using correlation data 430A from a plurality of patients who have been diagnosed with a particular condition. The system 400 may then create correlation nodes 435 for this condition profile that correspond to symptoms of that condition. By comparing a patient profile to the condition profile, the system 400 may find correlations between the patient profile and condition profile to deduce conditions for a particular patient. Therefore, in some preferred embodiments, the system 400 may automatically medically diagnose patients with conditions as correlation data 430A is updated in the system 400, which may assist medical professionals in better treating patients. In some preferred embodiments, the system 400 may use physician notes and observations to assist the physician in making a diagnosis. In other preferred embodiments, a user 405 may limit which correlation data 430A the system 400 may use to assist with making diagnosis and recommending treatments. For instance, a user 405 may want to exclude answers to questions submitted by a particular patient due to inconsistency between a patient's journal entries and a patients answer to said questions. This may grant users 405 exhaustive control over the way the system 400 assists in the diagnosis process.

A correlation node 435 may be defined as a defining characteristic of a PoV profile 430 derived from correlation data 430A within said PoV profile 430. In a preferred embodiment, correlation nodes 435 may comprise of one or more pieces of correlation data 430A, wherein each correlation node 435 within the plurality of correlation nodes 435 may represent at least one characteristic that may be associated with a particular PoV profile 430. For instance, a user 405 having correlation data 430A pertaining to several athletic activities may have a correlation node 435 having a characteristic that describes the user 405 as athletic. For instance, a user 405 having correlation data 430A pertaining to their children and those children's school activities may have a correlation node 435 having a characteristic that describes the user 405 as a parent of a child at the school in which the activities are taking place.

As mentioned previously, one preferred embodiment of the system 400 may comprise correlation nodes 435 sorted into groups 440 and subgroups. In another preferred embodiment, the system 400 may generate a group score 440A to determine how much each user 405 belongs to the group 440 and/or subgroup. For instance, a user 405 may who posts daily about the food that they eat may have several correlation nodes 435 pertaining to the food types that they eat. The user 405 may be placed by the system 400 in a “foodie” group comprising those correlation nodes 435, and the system 400 may then generate a group score 440A to determine how much the user 405 belongs in that group 440. The system 400 may use a combination of correlation nodes 435, groups 440, and group scores 440A to connect users 405 without the need for a contact between the users 405. In yet another preferred embodiment, the system 400 may weight correlation nodes 435 in way such that some characteristics of correlation nodes 435 have higher values when making connections than other characteristics. For instance, a correlation node 435 created from correlation data of a witnesses account of an event may be weighted more than a correlation node 435 created from correlation data comprising a hearsay account of an event.

In a preferred embodiments, the system 400 may use artificial intelligence (AI) techniques to create correlation nodes 435 from correlation data 430A of the system 400. The term “artificial intelligence” and grammatical equivalents thereof are used herein to mean a method used by the system 400 to correctly interpret and learn from data of the system 400 or a fleet of systems 400 in order to achieve specific goals and tasks through flexible adaptation. Types of AI that may be used by the system 400 include, but are not limited to, machine learning, neural network, computer vision, or any combination thereof. The system 400 preferably uses machine learning techniques to learn what historical information is relevant and which is not, wherein the instructions carried out by the processor 220 for said machine learning techniques are stored on the CRM 416. Machine learning techniques that may be used by the system 400 include, but are not limited to, regression, classification, clustering, dimensionality reduction, ensemble, deep learning, transfer learning, reinforcement learning, or any combination thereof

The system 400 may use more than one machine learning technique to generate correlation nodes 435 of the system 400. For instance, the system 400 may use a combination of natural language processing and reinforcement learning to learn the user's 405 writing style and deduce the most relevant information pertaining to a user's 405 interests from a user's 405 social media posts on a social media platform. Machine learning techniques may also be used to determine a mental state of a user 405 based on social media activity. In one preferred embodiment, the system 400 may recommend help to the user 405 if the system 400 determines that the user 405 is in danger due to their mental state. For instance, the system 400 may use supervised deep learning combined with results from computer-aided detection to deduce that a user 405 may be anxious or depressed and automatically connect the user 405 with a therapist. Over time, the system 400 may obtain more knowledge about a user's 405 mental state, allowing it to make more intelligent decisions about how to best address a user's 405 mental health needs.

As illustrated in FIG. 4, the system 400 may comprise a database 115 operably connected to the processor 220. The database 115 may be operably connected to the processor 220 via wired or wireless connection. In a preferred embodiment, the database 115 is configured to store correlation data 430A and image data 430B therein. Alternatively, the correlation data 430A and image data 430B may be stored on the CRM 416. The database 115 may be a relational database such that the correlation data 430A and image data 430B associated with each PoV profile 430 within the plurality of PoV profiles, at least in part, in one or more tables. Alternatively, the database 115 may be an object database such that correlation data 430A and image data 430B associated with each PoV profile 430 within the plurality of PoV profiles 430 may be stored, at least in part, as objects. In some instances, the database 115 may comprise a relational and/or object database and a server 110 dedicated solely to managing the correlation data 430A and image data 430B in the manners disclosed herein.

As mentioned previously, one embodiment of the system 400 may further comprise a computing device 300 operably connected to the processor 220. A computing device 300 may be implemented in a number of different forms, including, but not limited to, servers 110, multipurpose computers, mobile computers, etc. For instance, a computing device 300 may be implemented in a multipurpose computer that acts as a personal computer for a user 405, such as a laptop computer. For instance, components from a computing device 300 may be combined in a way such that a mobile computing device 350 is created, such as mobile phone. Additionally, a computing device 300 may be made up of a single computer or multiple computers working together over a network. For instance, a computing device 300 may be implemented as a single server 110 or as a group of servers 110 working together over and Local Area Network (LAN), such as a rack server 110 system 400. Computing devices 300 may communicate via a wired or wireless connection. For instance, wireless communication may occur using a Bluetooth, Wi-Fi, or other such wireless communication device.

In one preferred embodiment, the computing device 300 may comprise a geolocation device 412, such as a global positioning system (GPS) that allows the system 400 to receive geospatial data. Alternatively, a user 405 may manually input geospatial data corresponding to a specific geolocation in which the user 405 was located. For instance, a system 400 comprising a computing device 300 having a user interface 411 may allow a user 405 to input geospatial data using an input device such as a keyboard. For instance, a system 400 comprising a computing device 300 having a touch screen and a user interface 411 comprising a graphic information system (GIS) may allow a user 405 to select the geolocation on a map displayed via the touchscreen. As used herein, geospatial data may be spatial data including, but not limited to, numeric data, vector data, and raster data, or any combination thereof. Numeric data may be statistical data which includes a geographical component or field that can be joined with vector files so the data may be queried and displayed as a layer on a map in a GIS. Vector data may be data that has a spatial component, or X, Y coordinates assigned to it. Vector data may contain sets of points, lines, or polygons that are referenced in a geographic space. Raster data may be data in a .JPG, .TIF, .GIF or other picture file format. For instance, a map scanned in a flatbed scanner may be considered raster data.

In another preferred embodiment, the computing device 300 may comprise a camera 413, which a user 405 may use to capture image data 430B that may be stored within a PoV profile 430 either automatically at the time the picture was taken or at a later time as chosen by the user 405. For instance, a digital image of food eaten by the user 405 uploaded by a user 405 at the restaurant in which the user 405 is eating may be automatically stored within the user's 405 PoV profile 430 as soon as the user 405 captures the image data 430B. For instance, a digital image capture of a sunset on Mobile Bay may be uploaded to a user's 405 PoV profile 430 the day after the digital image was captured when selected by the user 405 via the user interface 411 of the system 400. In one preferred embodiment, when a digital image is received by the processor 220, the processor 220 may also receive geolocation data from the geolocation device 412. For instance, the system 400 may receive time data and geospatial data from a clock of the processor 220 and a geolocation device 412 of the computing device 300 when a digital image is captured using the camera 413 of the computing device 300. The system 400 may store this image data 430B in the user's 405 PoV profile 430 and use it to generate correlation nodes 435, groups 440, and group scores 440A that may be used to recommend social contacts.

In another preferred embodiment, the system 400 may parse/abstract correlation data 430A from a digital image taken by a user 405. For instance, a user 405 participating in the activity of diving may take a picture of an angel fish under water at a particular dive site, and the system 400 may automatically determine the dive site via digital signal processing and deduce that the user 405 participates in the hobby of diving. In an embodiment, the system 400 may use a machine learning technique to determine correlation data 430A within a digital image. For instance, pattern recognition or feature extraction may be used to determine correlation data 430A within a digital image. Pattern recognition methods may use labeled data that the system 400 may match to a digital image using algorithms to determine correlation data 430A. For instance, a user 405 may take a picture while watching a soccer game at local soccer stadium that the system 400 may recognize based structural features of the soccer stadium, uniforms of the teams, and logos on structures. Feature extraction methods may use algorithms to detect and isolate various desired portions or shapes of a digital image to determine correlation data 430A. Alternatively, the system 400 may use more than one machine learning technique to determine correlation data 430A from a digital image. For instance, if the system 400 fails to determine correlation data 430A using pattern recognition, the system 400 may subsequently attempt to determine correlation data 430A using feature extraction.

When correlation data 430A is parsed/abstracted from a digital image by the system 400, the system 400 may automatically save the correlation data 430A in a PoV profile 430. However, since there is a very large variation in the types of correlation data 430A that may be extracted by the system 400, one preferred embodiment of the system 400 may ask the user 405 if the correlation data 430A extracted from the digital image is correct. For instance, the system 400 may extract correlation data 430A that may indicate that the user 405 enjoys a particular artist based on a painting recognized by the system 400. The system 400 may then ask the user 405 if they enjoy that artist before saving the correlation data 430A in the user's 405 PoV profile 430. In addition, the correlation data 430A extracted from the digital image may be limited based on geospatial data 142G, though a user 405 may override this option via the user interface 411 if desired. For instance, the system 400 may recognize that the user's 405 geolocation would place them at a baseball field, and would limit and the types of correlation data 430A extracted from the digital image based on that condition. However, the baseball field may have been converted into a football field, which the system 400 may not be configured to recognized due to the condition based on the geolocation. Therefore, a user 405 may disable geolocation limited correlation data 430A extraction from digital images such that the system 400 may correctly extract correlation data 430A.

In an embodiment, the system 400 may further comprise a user interface 411. A user interface 411 may be defined as a space where interactions between a user 405 and the system 400 may take place. In a preferred embodiment, the interactions may take place in a way such that a user 405 may control the operations of the system 400, and more specifically, allow a user 405 to enter correlation data 430A and accept/deny social contacts recommended by the system 400. A user 405 may input instructions to control operations of the system 400 manually using an input device. For instance, a user 405 may choose to alter correlation data within their PoV profiles 430 by using an input device of the system 400, including, but not limited to, a keyboard, mouse, or touchscreen. A user interface 411 may include, but is not limited to operating systems, command line user interfaces, conversational interfaces, web-based user interfaces, zooming user interfaces, touch screens, task-based user interfaces, touch user interfaces, text-based user interfaces, intelligent user interfaces, and graphical user interfaces, or any combination thereof. The system 400 may present data of the user interface 411 to the user 405 via a display operably connected to the processor 220.

In a preferred embodiment, users 405 may access data of the system 400 via the user interface 411, which may be accomplished by causing the processor 220 to query the CRM 416 and/or database 115. The CRM 416 and/or database 115 may then transmit data back to the processor 220, wherein the processor 220 may present it to the user 405 via a display. This information may be presented to the user 405 in a way such that the user 405 may choose whether or not to accept a recommended social contact as well as view correlation nodes 435, groups 440, and group scores 440A generated by the system 400. The user interface 411 may also allow the user 405 to update historical information so that the system 400 may create more correlation nodes 435, groups 440, and group scores 440A that will allow the system 400 to make stronger social connections. The user interface 411 may also allow a user 405 to answer questions generated by the system 400 that may allow the system 400 to create correlation nodes 435, groups 440, and group scores 440A. Types of questions that may be asked by the system 400 include, but are not limited to, opinions about politics, hobbies, entertainment, work, food, or any combination thereof.

To prevent un-authorized users 405 from accessing data within of the system 400, the system 400 may employ a security method. As illustrated in FIG. 8, the security method of the system 400 may comprise a plurality of permission levels 800 that may allow a user 405 to view content 815, 835, 855 while simultaneously denying users 405 without appropriate permission levels 800 the ability to view said content 815, 835, 855. In a preferred embodiment, the data is stored within the database 115. To access the data stored within the database 115, users 405 may be required to make a request via a user interface 411. Access to the data within the database 115 may be granted or denied by the processor 220 based on verification of a requesting user's 805, 825, 845 permission level 800. If the requesting user's 805, 825, 845 permission level 800 is sufficient, the processor 220 may provide the requesting user 805, 825, 845 access to content 815, 835, 855 stored within the system 400. Conversely, if the requesting user's 805, 825, 845 permission level 800 is insufficient, the processor 220 may deny the requesting user 805, 825, 845 access to content 815, 835, 855 stored within the system 400. In an embodiment, permission levels 800 may be based on user roles 810, 830, 850 and administrator roles 870, as illustrated in FIG. 8. User roles 810, 830, 850 allow users 405 to access content 815, 835, 855 that a user 405 has uploaded and/or otherwise obtained through use of the system 400. Administrator roles 870 allow administrators 865 to access system 400 wide data, including managerial permissions, as well as assign new tasks to other users 405.

In an embodiment, user roles 810, 830, 850 may be assigned to a user 405 in a way such that a requesting user 805, 825, 845 may access PoV profiles 430 via a user interface 411. To access the data within the system 400, a user 405 may make a user request via the user interface 411 to the processor 220. In an embodiment, the processor 220 may grant or deny the request based on the permission level 800 associated with the requesting user 805, 825, 845 assigned via user roles 810, 830, 850. Only users 405 having appropriate user roles 810, 830, 850 or administrator roles 870 may access the content 815, 835, 855. For instance, as illustrated in FIG. 8, requesting user 1 805 has a permission level 800 to view user 1 content 815 whereas requesting user 2 825 has a permission level 800 to view user 1 content 815, user 2 content 835, and user 3 content 855. Alternatively, content 815, 835, 855 may be restricted in a way such that a user 405 may only view a limited amount of content 815, 835, 855. For instance, requesting user 3 845 may be granted a permission level 800 that only allows them to view user 3 content 855 related to a particular display. Therefore, the permission levels 800 of the system 400 may be assigned to users 405 in various ways without departing from the inventive subject matter described herein.

FIG. 9 provides a flow chart 900 illustrating certain, preferred method steps that may be used to carry out the method of generating a correlation node 435 and connecting users 405 via the correlation nodes 435. Step 905 indicates the beginning of the method. During step 910, the processor 220 may receive correlation data 430A from a user 405 via the user interface 411. Once received, the processor 220 may save the correlation data 430A within the user profile of the user 405 during step 915. The processor 220 may then analyze the correlation data 430A and generate correlation nodes 435 using AI techniques during step 920. In a preferred embodiment, machine learning, natural language processing, and temporal analysis are used to generate the correlation nodes 435. Once the correlation nodes 435 have been generated, the system 400 may perform a query to determine if the user 405 has any existing correlation nodes 435 within their user profile that may correspond with the new correlation nodes 435 during step 925.

Based on the results of this query, the system 400 may perform an action during step 930. If the processor 220 determines that a newly created correlation node 435 does correspond with a correlation node 435 within the user's 405 user profile, the system 400 may remove the correlation node 435 within the user profile during step 933 and subsequently the new correlation node 435 within the user profile during step 935. If the system 400 determines that a correlation node 435 does not correspond with a correlation node 435 within the user's 405 user profile, the system 400 may save the correlation node 435 within the user profile during step 935. Once the correlation nodes 435 have been saved within the user's 405 user profile, the system 400 may compare the correlation nodes 435 within the user's 405 user profile with that of correlation nodes 435 within other users' 405 user profiles during step 940 to determine if the user 405 has a connection with other users 405. Based on the results of this determination, the system 400 may perform an action during step 945. If the system 400 determines that the user 405 does have a connection with another user, the system 400 may recommend that the user 405 connect with the other user(s) 405 during step 947 before subsequently proceeding to the terminate method step 950. If the system 400 determines that the user 405 does not have a connection with any other users 405, the system 400 may proceed to terminate method step 950.

FIG. 10 provides a flow chart 1000 illustrating certain, preferred method steps that may be used to carry out the method of weighting correlation nodes 435. Step 1005 indicates the beginning of the method. During step 1010, the system 400 may obtain correlation data 430A from a user's PoV profile 430 during step 1015 and parse it for relevant information from which the system 400 may subsequently generate correlation nodes 435 using AI techniques during step 1020. Once the correlation nodes 435 have been generated, the system 400 may save the correlation node during step 1023 and subsequently perform a query to determine if any of the relevant information used to create the correlation nodes 435 correspond with keywords within a database 115 during step 1025, wherein said keywords have a score associated therewith that rate the importance. Based on the results of that query, the system 400 may perform an action during step during step 1030. If the system 400 determines that no relevant information matches a keyword, the system 400 may proceed to step 1035 and make connections based on said correlation nodes 435. If the system 400 determines that relevant information does match a keyword, the system 400 may associate the value of the keyword(s) in the database 115 with the correlation node 435 during step 1032.

Once the system 400 has associated values with the correlation nodes 435, the system 400 may divide the total value of each correlation node 435 with the total value assigned to all nodes during step 1034, which may allow the system 400 to rank the importance of a user's correlation node 435 based on other aspects within a user's user profile. The system 400 may then proceed to step 1035 and make connections with correlation nodes 435 of other PoV profiles 430. Because a PoV profile 430 may have nodes weighted differently due to the relevant information within the PoV profile 430, PoV profiles 430 may be correlated to each other differently. For instance, “User Profile A” may have multiple pieces of relevant information corresponding to a keyword within the database 115 whereas “User Profile B” may have one piece of relevant information corresponding to a keyword. As such, the correlation node(s) 435 of “User Profile B” corresponding to that particular piece of relevant information may be much more highly weighted when creating connections than any one of the correlation node(s) 435 of “User Profile A.” In some preferred embodiments, the system 400 may skip step 1034 and weigh the correlation nodes 435 based on the total assigned to each node. Once the system 400 has made connections using the correlation nodes 435, the system 400 may proceed to terminate method step 1040.

The subject matter described herein may be embodied in systems, apparati, methods, and/or articles depending on the desired configuration. In particular, various implementations of the subject matter described herein may be realized in digital electronic circuitry, integrated circuitry, specially designed application specific integrated circuits (ASICs), computer hardware, firmware, software, and/or combinations thereof. These various implementations may include implementation in one or more computer programs that may be executable and/or interpretable on a programmable system including at least one programmable processor, which may be special or general purpose, coupled to receive data and instructions from, and to transmit data and instructions to, a storage system, and at least one peripheral device.

These computer programs, which may also be referred to as programs, software, applications, software applications, components, or code, may include machine instructions for a programmable processor, and may be implemented in a high-level procedural and/or object-oriented programming language, and/or in assembly machine language. As used herein, the term “non-transitory computer-readable medium” refers to any computer program, product, apparatus, and/or device, such as magnetic discs, optical disks, memory, and Programmable Logic Devices (PLDs), used to provide machine instructions and/or data to a programmable processor, including a non-transitory computer-readable medium that receives machine instructions as a computer-readable signal. The term “computer-readable signal” refers to any signal used to provide machine instructions and/or data to a programmable processor. To provide for interaction with a user, the subject matter described herein may be implemented on a computer having a display device, such as a cathode ray tube (CRD), liquid crystal display (LCD), light emitting display (LED) monitor for displaying information to the user and a keyboard and a pointing device, such as a mouse or a trackball, by which the user may provide input to the computer. Displays may include, but are not limited to, visual, auditory, cutaneous, kinesthetic, olfactory, and gustatory displays, or any combination thereof.

Other kinds of devices may be used to facilitate interaction with a user as well. For instance, feedback provided to the user may be any form of sensory feedback, such as visual feedback, auditory feedback, or tactile feedback; and input from the user may be received in any form including, but not limited to, acoustic, speech, or tactile input. The subject matter described herein may be implemented in a computing system that includes a back-end component, such as a data server, or that includes a middleware component, such as an application server, or that includes a front-end component, such as a client computer having a graphical user interface or a Web browser through which a user may interact with the system described herein, or any combination of such back-end, middleware, or front-end components. The components of the system may be interconnected by any form or medium of digital data communication, such as a communication network. Examples of communication networks may include, but are not limited to, a local area network (“LAN”), a wide area network (“WAN”), metropolitan area networks (“MAN”), and the internet.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For instance, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, devices, and arrangements of the parts and method stages which have been described and illustrated in order to explain the nature of this inventive subject matter can be made without departing from the principles and scope of the inventive subject matter.

What is claimed is:

1) A system for making shared experiences networks comprising:
a computing device having a user interface,
wherein said user interface allows a user to choose input correlation data and create a user profile,
a processor operably connected to said computing device,
a geolocation device operably connected to said processor,
wherein said geolocation device is configured to collect geospatial data and transmit said geospatial data to said computing device,
a database operably connected to said processor,
wherein said database comprises keywords that have an importance value associated therewith,
a non-transitory computer-readable medium coupled to said processor,
wherein said non-transitory computer-readable medium contains instructions stored thereon, which, when executed by said processor, cause said processor to perform operations comprising:
receiving said input correlation data from said computing device,
generating a new correlation node using said input correlation data,
replacing an existing correlation node of said user profile with said new correlation node when said new correlation node and an existing correlation node overlap,
saving said new correlation node within said user profile when said new correlation node does not overlap said existing correlation node,
connecting said new correlation node with a plurality of existing correlation nodes within said user profile,
assigning importance values to said new correlation node and said plurality of existing correlation nodes using said keywords within said database,
creating a ranking of said new correlation node and said plurality of existing correlation nodes based on said importance values, and
making connections using said new correlation node and said plurality of existing correlation nodes based on said ranking.

2) The system of claim 1, wherein said new correlation node and said plurality of existing correlation nodes are sorted into a group and a subgroup, wherein said group and subgroup comprise a first characteristic and a second characteristic.

3) The system of claim 2, wherein a group score is generated for said group and said subgroup, wherein said group score allows said processor to determine how much said user belongs to said group and said subgroup.

4) The system of claim 3, wherein said new correlation node and said plurality of existing correlation nodes are weighted in way such that said first characteristic contributes more to said group score than said second characteristic.

5) The system of claim 3, wherein at least two of said new correlation node, plurality of existing correlation nodes, group, subgroup, and group scores are used to create a social connection between a first user and a second user, wherein said first user and said second user do not need an existing common contact for said social connection to be made, wherein said existing common contact is a third user that has said social connection with both said first user and said second user.

6) The system of claim 3, wherein at least two of said new correlation node, plurality of existing correlation nodes, group, subgroup, and group scores are used to create a medical diagnosis for a patient profile after comparison to a condition profile, wherein said condition profile is created using correlation data form a plurality of patient profiles having said medical diagnosis.

7) The system of claim 3, wherein at least two of said new correlation node, plurality of existing correlation nodes, group, subgroup, and group scores are used to create a primary lead from a plurality of lead profiles and a case profile, wherein each lead profile of said plurality of lead profiles contain correlation data pertaining to a particular lead, wherein said case profile contains correlation data pertaining to a particular investigation.

8) The system of claim 1, further comprising a camera operably connected to said processor, wherein said camera is configured to collect image data and transmit said image data to said computing device.

9) The system of claim 8, wherein said non-transitory computer-readable medium contains additional instructions, which, when executed by said processor, cause said processor to perform additional operations comprising:
receiving said image data from said camera,
extracting additional correlation data from said image data, and
generating a new correlation node using said image data and said additional correlation data.

10) A system for making shared experiences networks comprising:
a non-transitory computer-readable medium coupled to a processor and having instructions stored thereon, which, when executed by said processor, cause said processor to perform operations comprising:
receiving correlation data from a computing device,
generating a new correlation node using said correlation data,
replacing an existing correlation node of a user profile with said new correlation node when said new correlation node and an existing correlation node overlap,
saving said new correlation node within said user profile when said new correlation node does not overlap said existing correlation node,
connecting said new correlation node with a plurality of existing correlation nodes within said user profile,
assigning importance values to said new correlation node and said plurality of existing correlation nodes using keywords within a database,
creating a ranking of said new correlation node and said plurality of existing correlation nodes based on said importance values, and
making connections using said new correlation node and said plurality of existing correlation nodes based on said ranking.

11) The system of claim 10, wherein said new correlation node and said plurality of existing correlation nodes are sorted into a group and a subgroup, wherein said group and subgroup comprise a first characteristic and a second characteristic.

12) The system of claim 11, wherein a group score is generated for said group and said subgroup, wherein said group score allows said processor to determine how much said user profile belongs to said group and said subgroup.

13) The system of claim 12, wherein said new correlation node and said plurality of existing correlation nodes are weighted in way such that said first characteristic contributes more to said group score than said second characteristic.

14) The system of claim 12, wherein at least two of said new correlation node, plurality of existing correlation nodes, group, subgroup, and group scores are used to create a social connection between a first user and a second user, wherein said first user and said second user do not need an existing common contact for said social connection to be made, wherein said existing common contact is a third user that has said social connection with both said first user and said second user.

15) The system of claim 12, wherein at least two of said new correlation node, plurality of existing correlation nodes, group, subgroup, and group scores are used to create a medical diagnosis for a patient profile after comparison to a condition profile, wherein said condition profile is created using correlation data form a plurality of patient profiles having said medical diagnosis.

16) The system of claim 12, wherein at least two of said new correlation node, plurality of existing correlation nodes, group, subgroup, and group scores are used to create a primary lead from a plurality of lead profiles and a case profile, wherein each lead profile of said plurality of lead profiles contain correlation data pertaining to a particular lead, wherein said case profile contains correlation data pertaining to a particular investigation.

17) The system of claim 10, further comprising a camera operably connected to said processor,
wherein said camera is configured to collect image data and transmit said image data to said computing device.

18) The system of claim 17, wherein said non-transitory computer-readable medium contains additional instructions, which, when executed by said processor, cause said processor to perform additional operations comprising:
receiving said image data from said camera,
extracting additional correlation data from said image data, and
generating a new correlation node using said image data and said additional correlation data.

19) A method for making shared experiences networks comprising steps of:
receiving correlation data from a computing device,
generating a new correlation node using said correlation data,
replacing an existing correlation node of a user profile with said new correlation node when said new correlation node and an existing correlation node overlap,
adding said new correlation node to said user profile when said new correlation node does not overlap said existing correlation node,
connecting said new correlation node with a plurality of existing correlation nodes within said user profile,
assigning importance values to said new correlation node and said plurality of existing correlation nodes using keywords within a database,
creating a ranking of said new correlation node and said plurality of existing correlation nodes based on said importance values, and
making connections using said new correlation node and said plurality of existing correlation nodes based on said ranking.

20) The method of claim 19, further comprising the steps of:
receiving image data from a camera,
extracting additional correlation data from said image data, and
generating a new correlation node using said image data and said additional correlation data.
